SEQUENCE LISTING (26 sequences)

SEQ ID NO: 1 - RNA, 2001 nt, Homo sapiens
  source 1..2001: mol_type = genomic RNA
atggggcaaactaaaagtaaaattaaaagtaaatatgcctcttatctcagctttattaaaattcttttaaaaagagggggagttaaagtatctacaaaaaatctaatcaagctatttcaaataatagaacaattttgcccatggtttccagaacaaggaactttagatctaaaagattggaaaagaattggtaaggaactaaaacaagcaggtaggaagggtaatatcattccacttacagtatggaatgattgggccattattaaagcagctttagaaccatttcaaacagaagaagatagcgtttcagtttctgatgcccctggaagctgtttaatagattgtaatgaaaagacaaggaaaaaatcccagaaagaaacggaaagtttacattgcgaatatgtagcagagccggtaatggctcagtcaacgcaaaatgttgactataatcaattacaggaggtgatatatcctgaaacgttaaaattagaaggaaaaggtccagaattagtggggccatcagagtctaaaccacgagggacaagtcctcttccagcaggtcaggtgcccgtaacattacaacctcaaaagcaggttaaagaaaataagacccaaccgccagtagcctatcaatactggcctccggctgaacttcagtatcggccacccccagaaagtcagtatggatatccaggaatgcccccagcaccacagggcagggcgccataccctcagtcgcccactaggagacttaatcctacggcaccacctagtagacagggtagtgaattacatgaaattattgataaatcaagaaaggaaggagatactgaggcatggcaattcccagtaacgttagaaccgatgccacctggagaaggagcccaagagggagagcctcccacagttgaggccagatacaagtccttttcgataaaaatgctaaaagatatgaaagagggagtaaaacagtatggacccaactccccttatatgaggacattattagattccattgcttatggacatagactcattccttatgattgggagattctggcaaaatcgtctctctcaccctctcaatttttacaatttaagacttggtggattgatggggtacaagaacaggtccgaagaaatagggctgccaatcctccagttaacatagatgcagatcaactattaggaataggtcaaaattggagtactattagtcaacaagcattaatgcaaaatgaggccattgagcaagttagagctatcgtccttagagcctgggaaaaaatccaagacccaggaagcgcctgcccctcatttaatacagtaagacaaggttcaaaagagccctatcctgattttgtggcaaggctccaagatgttgctgaaaagtcaattgccgatgaaaaagcccgtaaggtcatagtggagttgatggcatatgaaaacgccaatcctgagtgtcaatcagccattaagtcattaaaagggaaggttcctgcaggatcagatgtaatctcagaatatgtaaaagtctgtgatggaatcggaggagctatgcataaagctatgcttatggctcaagcaataacaggagttgttttaggaggacaagttagaacatttggaggaaaatgttataattgtggtcaaattggtcacttaaaaaagagttgcccagtcttaaacaaacagaatataactattcaagcaactacaacaggtagagagccacctgacttatgtccaagatgtaaaaaaggaaaacattgggctagtctatgtcgttctaaatttgataaaaatgggcaaccattgtcgggaaacgagcaaaggggccagcctcaggccccacaacaaactggggcattcccaattcagccatttgttcctcagggttttcagggacaacaacccccactgtcccaagtgtttcagggaataagccagttaccacaatacaacaattgtcccctgccacaagcagcagtgcagcagtag

SEQ ID NO: 2 - RNA, 21 nt, synthetic construct
  source 1..21: mol_type = other RNA
cagtttctgatgcccctggaa

SEQ ID NO: 3 - RNA, 20 nt, synthetic construct
  source 1..20: mol_type = other RNA
aatactggcctccggctgaa

SEQ ID NO: 4 - RNA, 20 nt, synthetic construct
  source 1..20: mol_type = other RNA
tgtgatggaatcggaggagc

SEQ ID NO: 5 - RNA, 20 nt, synthetic construct
  source 1..20: mol_type = other RNA
gggtggccgatactgaagtt

SEQ ID NO: 6 - RNA, 21 nt, synthetic construct
  source 1..21: mol_type = other RNA
ccagaatctcccaatcataag

SEQ ID NO: 7 - RNA, 20 nt, synthetic construct
  source 1..20: mol_type = other RNA
tgagagggtgagagagacga

SEQ ID NO: 8 - RNA, 22 nt, synthetic construct
  source 1..22: mol_type = other RNA
cttggagccttgccacaaaatc

SEQ ID NO: 9 - RNA, 20 nt, synthetic construct
  source 1..20: mol_type = other RNA
ggacagtgggggttgttgtc

SEQ ID NO: 10 - RNA, 20 nt, synthetic construct
  source 1..20: mol_type = other RNA
aaacacttgggacagtgggg

SEQ ID NO: 11 - protein, 666 aa, unidentified
MGQTKSKIKSKYASYLSFIKILLKRGGVKVSTKNLIKLFQIIEQFCPWFPEQGTLDLKDWKRIGKELKQAGRKGNIIPLTVWNDWAIIKAALEPFQTEEDSVSVSDAPGSCLIDCNEKTRKKSQKETESLHCEYVAEPVMAQSTQNVDYNQLQEVIYPETLKLEGKGPELVGPSESKPRGTSPLPAGQVPVTLQPQKQVKENKTQPPVAYQYWPPAELQYRPPPESQYGYPGMPPAPQGRAPYPQSPTRRLNPTAPPSRQGSELHEIIDKSRKEGDTEAWQFPVTLEPMPPGEGAQEGEPPTVEARYKSFSIKMLKDMKEGVKQYGPNSPYMRTLLDSIAYGHRLIPYDWEILAKSSLSPSQFLQFKTWWIDGVQEQVRRNRAANPPVNIDADQLLGIGQNWSTISQQALMQNEAIEQVRAIVLRAWEKIQDPGSACPSFNTVRQGSKEPYPDFVARLQDVAEKSIADEKARKVIVELMAYENANPECQSAIKSLKGKVPAGSDVISEYVKVCDGIGGAMHKAMLMAQAITGVVLGGQVRTFGGKCYNCGQIGHLKKSCPVLNKQNITIQATTTGREPPDLCPRCKKGKHWASLCRSKFDKNGQPLSGNEQRGQPQAPQQTGAFPIQPFVPQGFQGQQPPLSQVFQGISQLPQYNNCPLPQAAVQQ

SEQ ID NO: 12 - RNA, 25 nt, synthetic construct
  source 1..25: mol_type = other RNA
atagaacaattttgcccatggtttc

SEQ ID NO: 13 - RNA, 21 nt, synthetic construct
  source 1..21: mol_type = other RNA
tcattccacttacagtatgga

SEQ ID NO: 14 - RNA, 25 nt, synthetic construct
  source 1..25: mol_type = other RNA
agatagcgtttcagtttctgatgcc

SEQ ID NO: 15 - RNA, 25 nt, synthetic construct
  source 1..25: mol_type = other RNA
gaaaataagacccaaccgccagtag

SEQ ID NO: 16 - RNA, 20 nt, synthetic construct
  source 1..20: mol_type = other RNA
tatggacccaactcccctta

SEQ ID NO: 17 - RNA, 21 nt, synthetic construct
  source 1..21: mol_type = other RNA
cttatgattgggagattctgg

SEQ ID NO: 18 - RNA, 24 nt, synthetic construct
  source 1..24: mol_type = other RNA
aaattggagtactattagtcaaca

SEQ ID NO: 19 - RNA, 25 nt, synthetic construct
  source 1..25: mol_type = other RNA
tgtcaatcagccattaagtcattaa

SEQ ID NO: 20 - RNA, 25 nt, synthetic construct
  source 1..25: mol_type = other RNA
ctactggcggttgggtcttattttc

SEQ ID NO: 21 - RNA, 21 nt, synthetic construct
  source 1..21: mol_type = other RNA
taataatgtcctcatataagg

SEQ ID NO: 22 - RNA, 26 nt, synthetic construct
  source 1..26: mol_type = other RNA
gcctcattttgcattaatgcttgttg

SEQ ID NO: 23 - RNA, 25 nt, synthetic construct
  source 1..25: mol_type = other RNA
ttaatgacttaatggctgattgaca

SEQ ID NO: 24 - RNA, 20 nt, synthetic construct
  source 1..20: mol_type = other RNA
tgaccaatttgaccacaatt

SEQ ID NO: 25 - RNA, 24 nt, synthetic construct
  source 1..24: mol_type = other RNA
tgcccatttttatcaaatttagaa

SEQ ID NO: 26 - RNA, 20 nt, synthetic construct
  source 1..20: mol_type = other RNA
gccccagtttgttgtggggc

(19) United States
(12) Patent Application Publication
(10) Pub. No.: US 2025/0066854 A1
(43) Pub. Date: Feb. 27, 2025

(54) BIOMARKER METHOD FOR EARLY DIAGNOSIS OF TYPE 1 DIABETES

(71) Applicants: Yang Dai, Poway, CA (US); Wen-Yuan Hu, San Diego, CA (US)

(72) Inventors: Yang Dai, Poway, CA (US); Wen-Yuan Hu, San Diego, CA (US)

(21) Appl. No.: 18/792,021

(22) Filed: Aug. 1, 2024

Related U.S. Application Data
(60) Provisional application No. 63/578,452, filed on Aug. 24, 2023.

(51) Int. Cl. C12Q 1/6883

(52) U.S. Cl. CPC C12Q 1/6883; C12Q 2600/156; C12Q 2600/158

(57) ABSTRACT

A method of early detection of Type 1 diabetes uses a quantitative assay to measure biomarkers of autoimmune diseases at specific short regions of a gene sequence. The assay uses amplicon deep sequencing to quantify individual variants of the ERV Group Antigen (Gag) gene associated with an overactive immune response.

### CROSS-REFERENCE TO RELATED APPLICATION

This application claims the benefit of priority of U.S. provisional application No. 63/578,452, filed Aug. 24, 2023, the contents of which are herein incorporated by reference.

### BACKGROUND OF THE INVENTION

The present invention relates to autoimmune diseases and, more particularly, to a biomarker method for early diagnosis of type 1 diabetes.

Autoimmune diseases, including type 1 diabetes (T1D), are a group of diseases caused by overreactive immune responses to abnormal genetic or environmental triggers. Typically, the primary trigger of a specific autoimmune disease is unknown, making it impossible to prevent these diseases. There is no cure for T1D. Therefore, early diagnosis of high-risk children is critical for prevention and treatment. Screening children for risk of T1D is crucial for identifying high-risk and pre-diabetic children for preventing complications such as diabetes ketoacidosis, a life-threatening condition of T1D.

Current screening methods rely on genetic and autoantibody tests. Although some of these tests could provide significant information about disease progression in well-controlled research trials, their future remains unclear. Also, caveats and limitations of these methods prevent their use in clinical practice.

Genetic testing is complex and inaccurate. Various genes participating in multiple pathways of the immune system contribute to overreactive immune responses, making it difficult to target one specific gene for treatment. Nevertheless, it is very clear that this group of diseases is commonly associated with the class II major histocompatibility complex (MHC) genes or human leukocyte antigen (HLA) in humans. The MHC or HLA molecules function to present peptide antigens to stimulate T cells. A plausible explanation for this genetic association is that specific self-antigens become abnormal and stimulatory for autoreactive T cells; such antigens are called autoantigens. Following this lead, specific autoantigens for each of the autoimmune diseases have been identified, and their cognate immune elements, such as autoantibodies and autoreactive T cells, are targeted for diagnosis or treatment. Intriguingly, these autoantigens, however, do not have unique features that are linked to the MHC or HLA, and treatment strategies based on these autoantigens have not been successful in clinics, suggesting that new antigens remain to be discovered.

T1D is the prototype of an autoimmune disease that shares key characteristics of this group of disease, including a strong genetic association with MHC or HLA genes, an overreactive immune system, and the development of autoantibodies specific to the autoantigens. The T1D-susceptible HLA genes, HLA-DR3/4 and HLA-DQ2/8, and the autoantigens e.g. insulin, Glutamic acid decarboxylase 65 (GAD65), and Islet Antigen 2 (IA-2) have been discovered. One of the animal models for T1D, non-obese diabetic (NOD) mice, has been extensively studied worldwide. As a result, the understanding of T1D genetics has led to genetic screening methods that can differentiate between high-risk and low-risk children. However, the process from the initial triggering of the autoimmune response to the complete destruction of pancreatic islets and the onset of diabetes can take several years. Not all high-risk children will experience the triggering of autoimmune responses, and many of the high-risk children, even with autoantibodies, do not develop diabetes. Unfortunately, the genetic testing could not be used to monitor this process and cannot predict who will become diabetic.

Autoantibody testing could offer helpful information on disease progression. The known autoantigens permit developing autoantibody-based diagnostic assays that aid in the diagnosis of pre-diabetic patients. However, while this method has been developed for over 30 years, its usage remains as research only, not a recommended clinical practice. This is because out of many different types of autoantibodies, there is no clear rule which is associated with higher risk or early signs of T1D. Also, even in the same patient, the types and/or tiers of autoantibodies change during the course of disease progression. More importantly, the reagents for autoantibody testing vary in quality and are difficult to standardize, which prevents this method from use in clinical practice.

As can be seen, there is an urgent need to identify new biomarkers that can accurately predict the different early stages and the disease progression before the onset of diabetes to facilitate disease prevention.

### SUMMARY OF THE INVENTION

In one aspect of the present invention, a method utilizes a serum biomarker for early detection of type 1 diabetes (T1D). This present invention includes a methodology for developing a quantitative assay to measure the association between endogenous retroviruses (ERVs) and autoimmune diseases, specifically T1D. The methodology uses deep sequencing to quantify individual sequence variants of the ERV Group Antigen (Gag) gene, identified herein as SEQ ID: 1, overcoming previous challenges in measuring ERV gene expression due to the presence of numerous highly similar copies of ERV gene sequences in the genome. The Gag gene has four major structural domains: matrix (MA), capsid (CA), nucleocapsid (NC), and an unstructured C-terminus peptide (p6). The method of the present subject matter targets specific short regions of the gene, preferably the conserved ones, enabling the adoption of a cost-effective sequencing technology, amplicon deep sequencing, for quantification purposes. Additionally, the invention incorporates a novel data analysis approach that focuses solely on sequences coding for open reading frames (ORFs), enhancing the method's sensitivity. The ribonucleic acid (RNA)/deoxyribonucleic acid (DNA)-based immunological biomarker of this assay offers advantages over protein-based T1D biomarkers, such as autoantibodies, including the reproducibility of the assay by different laboratories and the accuracy in diagnosing pre-diabetic stages before disease onset.

These and other features, aspects and advantages of the present invention will become better understood with reference to the following drawings, description, and claims.

### BRIEF DESCRIPTION OF THE DRAWINGS

FIGS. 1, 2, and 3 are a schematic diagram illustrating steps of a diagnostic method according to an embodiment of the present invention;

FIG. 4A is a schematic illustrating a genetic sequence and a chromatogram thereof;

FIG. 4B is a chart illustrating experimentally obtained percentages of sequence variants from mouse strains with different T1D risks;

FIG. 4C is another chart illustrating experimentally obtained percentages of sequences coding for ORFs;

FIG. 5A is another chart illustrating experimentally obtained percentages of sequence variants by age in the NOD mouse model;

FIG. 5B is another chart illustrating experimentally obtained percentages of sequences coding for ORFs by age;

FIG. 5C is another chart illustrating experimentally obtained consensus score by age;

FIG. 6A is another chart illustrating experimentally obtained percentages of sequences coding for ORFs by age in a longitudinal study of NOD mice;

FIG. 6B is another chart illustrating experimentally obtained consensus score by age;

FIG. 7A is a chart illustrating experimentally obtained correlation of sequences coding for ORFs to age of T1D patients;

FIG. 7B is another chart illustrating experimentally obtained correlation of sequences coding for ORFs to the development of autoantibodies; and

FIG. 7C is another chart illustrating experimentally obtained correlation of sequences coding for ORFs to oral glucose tolerance of T1D patents.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, an embodiment of the present invention provides a method utilizing a serum biomarker, derived from analyzing endogenous retroviral sequences, for determining potential risk for of type 1 diabetes (T1D), or early detection of T1D, or for monitoring T1D disease progression.

As used herein, the term “deep sequencing method”, also known as “massively parallel sequencing”, “ultra-high throughput sequencing”, and “next generation sequencing”, refers to a method comprising sequencing a genomic region multiple times. In some embodiments, sequencing is performed at least 100 times, preferably at least 1000 times, more preferably at least 10,000 times, even more preferably at least 100,000 times, and most preferably at least 1,000,000 times. Deep sequencing enables detection and quantification of a target comprising as little as 1% of an original sample.

The term “identity” is sometimes used herein to describe the nucleotide content and order of a sequence. Primers bind to ERV matter to prepare cDNA and amplify it to obtain enough signals for a sequencing step. ERV matter is highly heterogeneous and contains many slightly different sequence variants. The primers may be optimized by replacing a small number of nucleotides in some cases. Any primer sequences that share more than 80% identity with a sequence shares at least 80% of the nucleotides in the sequence in the same order. Note that each position in the sequence could have 4 nucleotide options: A/G/T/C.

Approximately 5-8% of genome sequences consist of ancient retroviruses that integrated into the host genome through germ line infection. These retroviruses, known as endogenous retroviruses (ERVs), have a similar genetic organization to retroviruses, including long terminal repeat (LTR), Group Antigens (Gag), Pro, Pol, and Envelope (Env) genes. Retroviruses have a preference for infecting lymphocytes, leading to coevolution with the adaptive immune system. While ERVs usually remain inactive, there are instances where abnormal ERV activation can occur, resulting in persistent stimulation of the adaptive immune system and triggering autoimmunity. Therefore, genes involved in controlling ERV activation or immune responses are integral to ERV-linked autoimmune diseases. One such gene is the MHC, which is highly polymorphic and associated with both murine leukemia retrovirus (MuLV) infection and autoimmune disease. See Hasenkrug, K. J., et al. (1997), Immunity to retroviral infection: the Friend virus model, Proc Natl Acad Sci U S A 94, 7811-7816 and Todd, J. A., et al. (1987), HLA-DQ beta gene contributes to susceptibility and resistance to insulin-dependent diabetes mellitus, Nature 329, 599-604, the disclosures of which are incorporated herein in their entireties. ERV antigens can act as “superantigens” for T cells as discussed in Marrack, P., et al. (1993), The bacterial and mouse mammary tumor virus superantigens; two different families of proteins with the same functions, Immunol Rev 131, 79-92, the disclosure of which is incorporated herein in its entirety.

ERVs, and human endogenous retroviruses (HERVs) in particular, are known to relate to human diseases, including T1D. These viral elements in our genome are “fixed”, meaning that they are unable to produce live, infectious retroviruses due to mutations/deletions/insertions, etc. of the viral genes. However, certain parts of the viruses, such as the Gag gene, may be resistant and retain complete or partial open-reading frames. This allows them to retain the capacity to produce certain viral gene products that can affect cellular and molecular reactions, particularly antiviral immune responses, and thus cause autoimmune diseases such as T1D. One HERV-K family virus, HERV-K(C4), seems to inhibit the development of T1D; see Mason, M. J., et al. (2014), Low HERV-K(C4) copy number is associated with type 1 diabetes, Diabetes 63, 1789-1795. More recently, it has been suggested that the Env protein of HERV-W is involved in T1D pathogenesis, as discussed by Levet. S., et al. (2017) in An ancestral retroviral protein identified as a therapeutic target in type 1 diabetes, JCI insight 2. In NOD mice, an ERV similar to Murine Leukemia Virus (MuLV) has been identified. See Gaskins, H. R., et al. (1992), Beta cell expression of endogenous xenotropic retrovirus distinguishes diabetes-susceptible NOD/Lt from resistant NON/Lt mice, J Clin Invest 90, 2220-2227; Bashratyan, R., et al. (2017). T1D pathogenesis is modulated by spontaneous autoimmune responses to endogenous retrovirus antigens in NOD mice, Eur J Immunol 47, 575-584; and Dai, Y. D., et al. (2020), Endogenous retrovirus Gag antigen and its gene variants are unique autoantigens expressed in the pancreatic islets of non-obese diabetic mice, Immunol Lett 223, 62-70. The disclosures of Perron et al., Mason et al., Levet et al., Gaskins et al., Bashratyan et al., and Dai et al. are incorporated herein by reference in their entireties. Overall, these findings indicate that the expression of endogenous retroviral genes and antigens varies between individuals susceptible to T1D and those who are resistant.

HERV-K is the most recent retrovirus to infect the human germ line. Members of HERV-K viruses are also known as Human MMTV-like 2 (HML-2). This family of HERVs is unique to humans and is not present in our closest primate relatives. This suggests that the virus is a relatively recent occurrence in evolution and is currently highly active. The human genome contains about 100 distinct copies of HERV-K that are closely related in terms of sequence similarity. While none of these HERV-Ks can generate infectious retroviruses, many of them have not yet been fully deactivated or fixed. The genome of an endogenous retrovirus consists of at least four fundamental genes: Gag, Pro, Pol, and Env. Certain viral elements, such as Gag, still have functional units that contribute to various processes in the host's development and physiology. In some cases, these elements can disrupt normal processes, leading to conditions like autoimmune diseases and T1D.

Gag is the most conserved gene across different members of the HERV-K family. Many HERV-K viruses have a complete Gag gene that codes for a full-length open reading frame (ORF), enabling production of a functional Gag protein. The Gag molecule possesses specific biochemical characteristics that allow it to interact with immune cells, particularly T cells. This interaction involves the exploitation of the intracellular exosome secretion machinery to aid in its release as virus-like particles and subsequent stimulation of immune cells. Applicants have discovered that the release of Gag, along with exosomes, stimulates autoreactive T cells to produce Interferon gamma (IFN-g), an important pro-inflammatory cytokine that is responsible for the destruction of islets and the onset of T1D. The sole target gene to be detected and analyzed in the present method may be the ERV Gag gene. Once identified, a specific region(s) of the ERV Gag gene can be analyzed for sequence variants using a deep-sequencing technology.

We have surprisingly discovered that the numbers of Gag variants, ORFs, and specific nucleotides and amino acid residues are correlated with disease progression. Without being bound by theory, Gag could be a primary autoantigen that drives the autoimmune responses in T1D, and thus Gag may be a therapeutic target.

Most Gag genes in HERV-K viruses are approximately 2000 base pairs (bp) long. Amplicon deep sequencing, a cost-effective deep sequencing method, has a size limit of under 500 bp. In embodiments, the capsid region within Gag is sequenced because it is relatively more conserved compared to other regions, which generally results in better analysis outcomes, particularly in determining ORFs.

Generally, the method comprises analyzing ERVs, particularly of the Gag gene and its products to estimate or predict risk for T1D. A challenge in examining the ERVs is that there are thousands of copies of ERVs in the genome, and many of them are very similar in the nucleotide (nt) sequences. High-throughput gene expression analysis is nonspecific for ERV genes and thus reduces sensitivity in analyzing their gene variants; also, it is costly and requires great effort in analyzing data. The method of the present subject matter overcomes the complexity of ERVs and the challenges in DNA sequencing by performing a cost-effective, targeted deep sequencing analysis, which focuses on selected regions within the Gag gene to reveal its sequence complexity and relation to disease. The method estimates the risk of T1D by measuring the levels of gene expression and gene variants of the Gag gene and its protein products.

The present method is an RNA-based molecular technology that measures the expression of ERVs in blood serum to predict the risk and progress toward T1D onset. The method uses standard reagents and protocols. It measures one molecular target, whereas prior genetic and autoantibody tests measure multiple targets or use non-standardized reagents. More importantly, because the method is an RNA-based technology and capable of identifying minor changes at nucleotide sequences, it is more sensitive and reproducible than prior methods.

Finally, in addition to T1D, this invention can be used to assess and/or detect other autoimmune diseases that may also relate to ERVs, such as lupus, arthritis, multiple sclerosis, etc.

In some embodiments, computer programming may be used to reduce errors and times in estimating T1D risk based on the sequencing results of the specific regions of the ERV Gag gene. A software product may be made to analyze ERV genes and identify DNA sequence marker(s) to estimate risk for T1D.

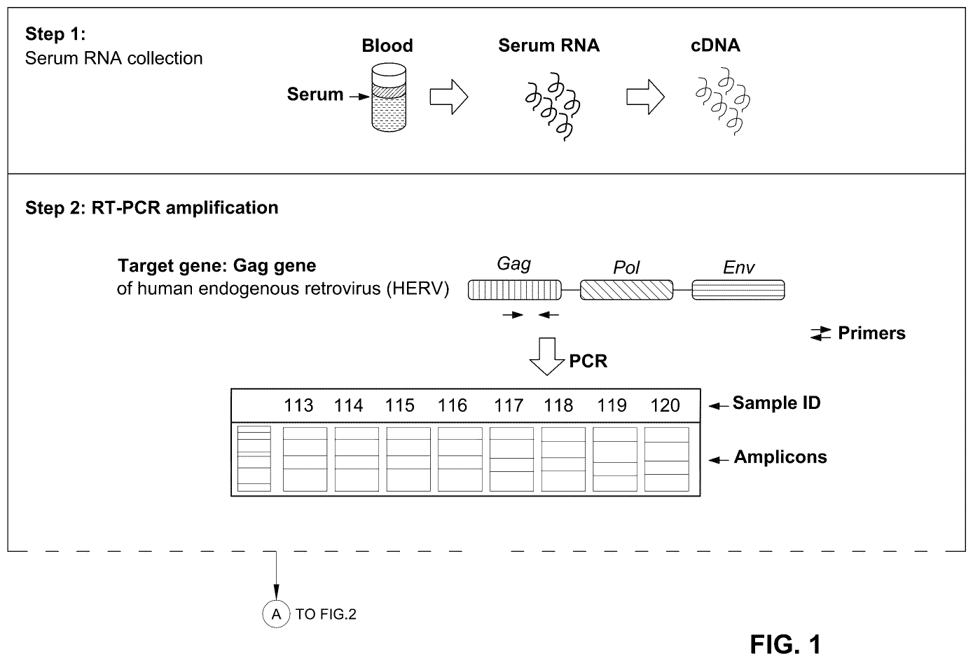

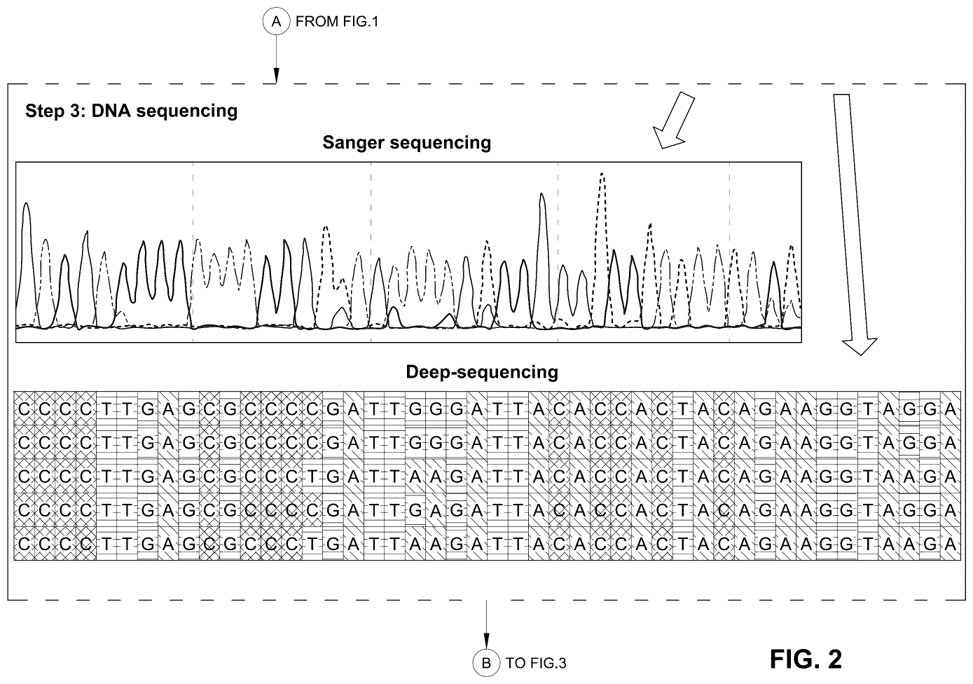

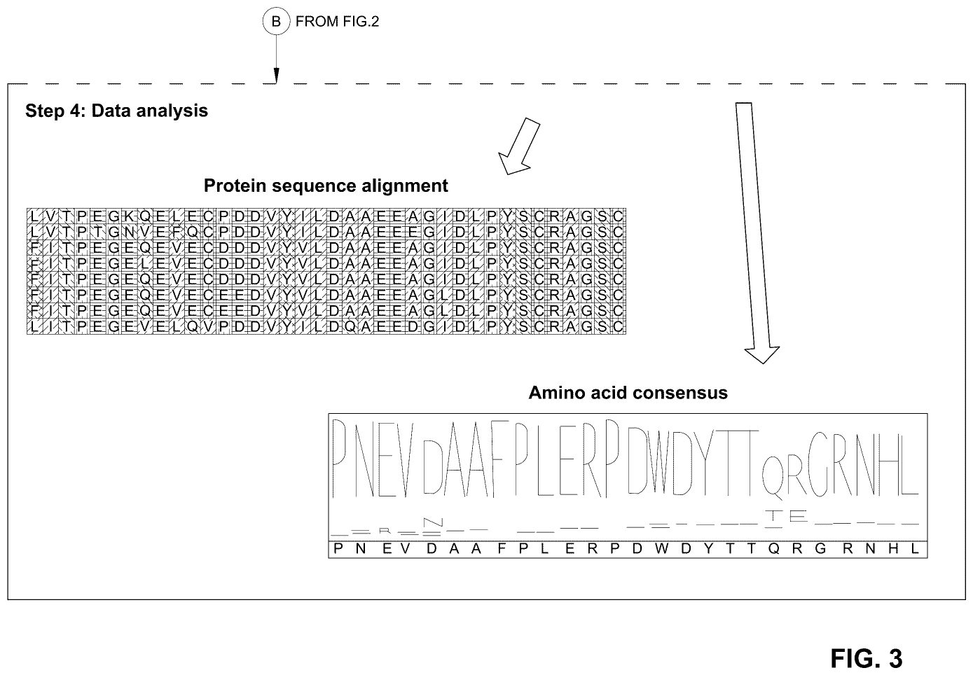

Referring to FIGS. 1-3, 4A, 4B, 4C, 5A, 5B, 5C, 6A, 6B, 7A, 7B, and 7C, FIG. 1 illustrates a method for assessing risk of and/or detecting T1D according to an embodiment of the present invention. The method includes sample preparation (Step 1), PCR amplification (Step 2), DNA sequencing (Step 3, see FIG. 2), and data analysis (Step 4, see FIG. 3). Step 1, sample preparation, includes processing a patient's blood according to methods known in the art to extract serum for further processing, as shown in FIG. 1. RNA is then isolated from the extracted serum by known RNA purification methods. A DNA denaturation step may be employed to minimize contamination from DNA in the serum. Complementary DNA (cDNA) is synthesized by reverse transcription (RT) from the isolated RNA, including the ERV genetic matter. The RNA may be degraded enzymatically into RNA fragments with a reverse transcriptase in the presence of a buffer, deoxynucleotide triphosphates (dNTPs), dithiothreitol (DTT), ribonuclease (RNase) inhibitor, nuclease-free water, and primers. To synthesize cDNA from the RNA fragments, the steps of primer annealing, DNA polymerization, and enzyme deactivation are performed. Advantageously, cDNA can be utilized directly as a template for Polymerase Chain Reaction (PCR) amplification.

Step 2 targets specific HERV genes to generate PCR amplicons that serve as disease markers. A specifically designed primer is utilized to bind to targeted conserved regions throughout the ERV Gag gene, with a higher concentration in the capsid region. These primers target specific regions within Gag to amplify the ERV matter in the cDNA and affect the sensitivity and accuracy of the method. In embodiments, the primer design strategy involves aligning multiple HERV-K Gag genes to identify conserved regions that can be targeted for primer binding. In embodiments, the primer is designed to specifically target the capsid region of the Gag gene. However, other regions within Gag may also be targeted. Table 1 shows a list of primers suitable for the method disclosed herein. Advantageously, the capsid region is chosen for binding because of the higher concentration of conserved regions relative to other regions of the ERV.

TABLE 1

| FORWARD PRIMERS: | REVERSE PRIMERS: |
|---|---|
| SEQ ID NO: 2; K-F308: | SEQ ID NO: 5; K-R669: |
| 5-CAGTTTCTGATGCCCCTGGAA-3′ | 5′-GGGTGGCCGATACTGAAGTT-3′ |
| SEQ ID NO: 3; K-F632: | SEQ ID NO: 6; K-R1040: |
| 5′-AATACTGGCCTCCGGCTGAA-3′ | 5′-CCAGAATCTCCCAATCATAAG-3′ |
| SEQ ID NO: 4; K-F1537: | SEQ ID NO: 7; K-R1085: |
| 5′-TGTGATGGAATCGGAGGAGC-3′ | 5′-TGAGAGGGTGAGAGAGACGA-3′ |
| SEQ ID NO: 12; K-F148: | SEQ ID NO: 8; K-R1357: |
| 5′- ATAGAACAATTTTGCCCATGGTTTC-3′ | 5′-CTTGGAGCCTTGCCACAAAATC-3′ |
| SEQ ID NO: 13; K-F247: | SEQ ID NO: 9; K-R1926: |
| 5′- TCATTCCACTTACAGTATGGA-3′ | 5′-GGACAGTGGGGGTTGTTGTC-3′ |
| SEQ ID NO: 14; K-F321: | SEQ ID NO: 10; K-R1935: |
| 5′- AGATAGCGTTTCAGTTTCTGATGCC-3′ | 5′-AAACACTTGGGACAGTGGGG-3′ |
| SEQ ID NO: 15; K-F625: | SEQ ID NO: 20; K-R601: |
| 5′-GAAAATAAGACCCAACCGCCAGTAG-3′ | 5′- CTACTGGCGGTTGGGTCTTATTTTC-3′ |
| SEQ ID NO: 16; K-F992: | SEQ ID NO: 21; K-R988: |
| 5′- TATGGACCCAACTCCCCTTA-3′ | 5′- TAATAATGTCCTCATATAAGG-3′ |
| SEQ ID NO: 17; K-F1060: | SEQ ID NO: 22; K-R1219: |
| 5′- CTTATGATTGGGAGATTCTGG-3′ | 5′- GCCTCATTTTGCATTAATGCTTGTTG-3′ |
| SEQ ID NO: 18; K-F1223: | SEQ ID NO: 23; K-R1462: |
| 5′- AAATTGGAGTACTATTAGTCAACA-3′ | 5′- TTAATGACTTAATGGCTGATTGACA-3′ |
| SEQ ID NO: 19; K-F1486: | SEQ ID NO: 24; K-R1642: |
| 5′- TGTCAATCAGCCATTAAGTCATTAA-3′ | 5′- TGACCAATTTGACCACAATT-3′ |
| | SEQ ID NO: 25; K-R1788: |
| | 5′- TGCCCATTTTTATCAAATTTAGAA-3′ |
| | SEQ ID NO: 26; K-R1849: |
| | 5′- GCCCCAGTTTGTTGTGGGGC-3′ |

In embodiments, binding of the cDNA and primer results in the production of an amplicon for deep sequencing analysis. In embodiments, the resulting amplicon is the nt838-1197 amplicon. Advantageously, the selection of amplicons for deep sequencing provides a cost-effective mechanism for DNA sequencing.

At step 3, two cost-effective sequencing analysis methods, regular Sanger sequencing and amplicon deep-sequencing (a quantitative means of determining sequence variations), are utilized. To determine sequence variants, short amplicons within the Gag gene are needed. By aligning different genomic Gag sequences, conserved regions of the Gag gene, primers designed to produce a PCR amplicon, were identified. Using Sanger sequencing according to methods well known in the art, variants on the chromatograms can be observed. Deep sequencing is utilized to quantify individual sequence variants within the nt838-1197 amplicon. For each sample, at least 200,000 sequences may be obtained, with analysis performed on a minimum of 50,000 matched sequences.

At step 4, nucleotide sequences generated from the amplicon deep sequencing step and their corresponding translated amino acid sequences are analyzed to quantify the scores needed to predict disease risk and progression. In embodiments, three different scores are used: the number of different sequences (variants), the sequences that encode an ORF, and the consensus sequence of specific nucleotides or amino acid residues.

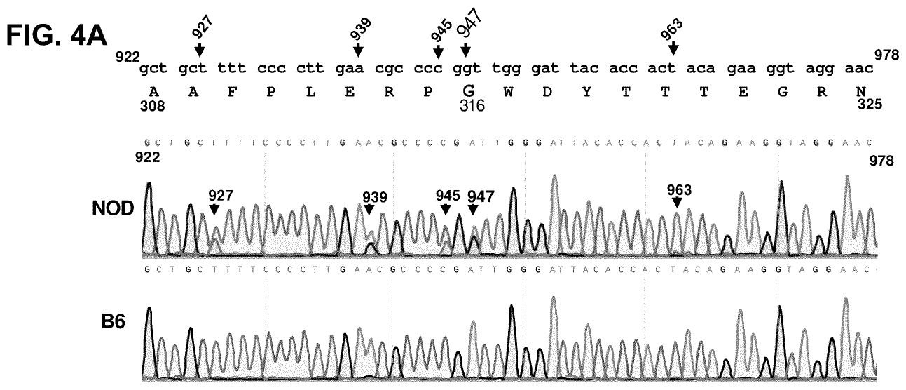

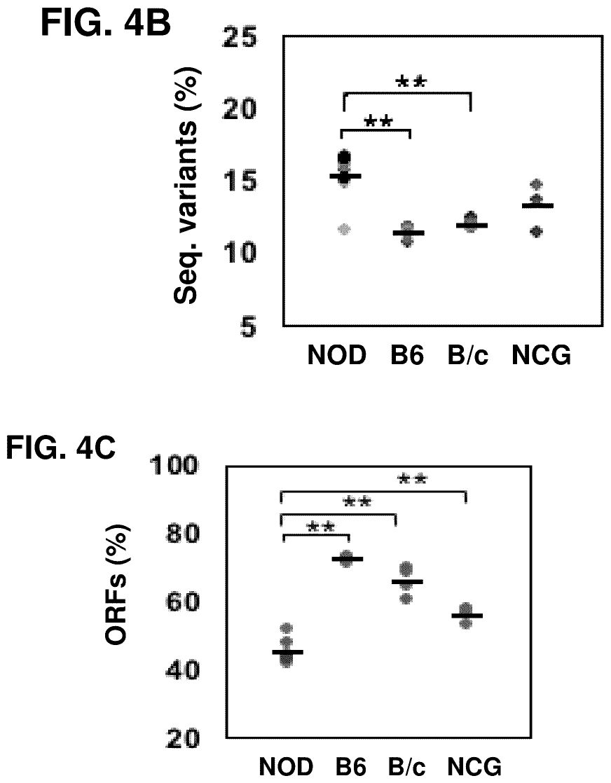

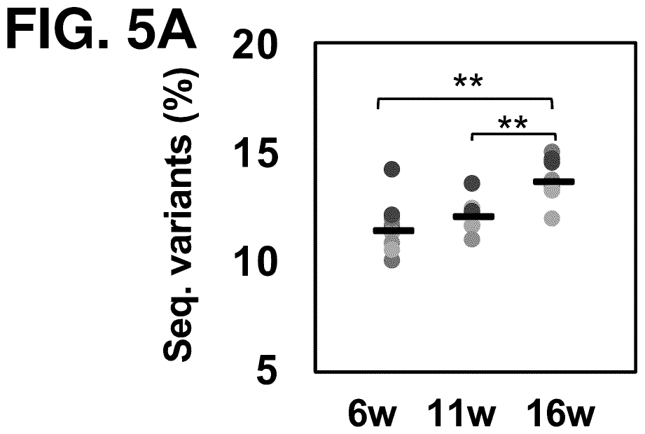

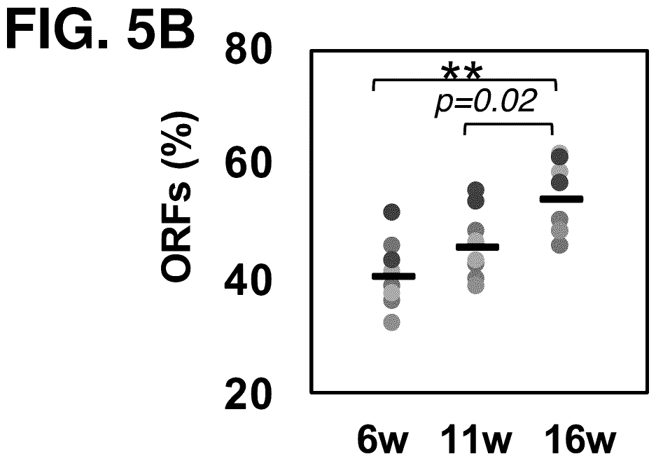

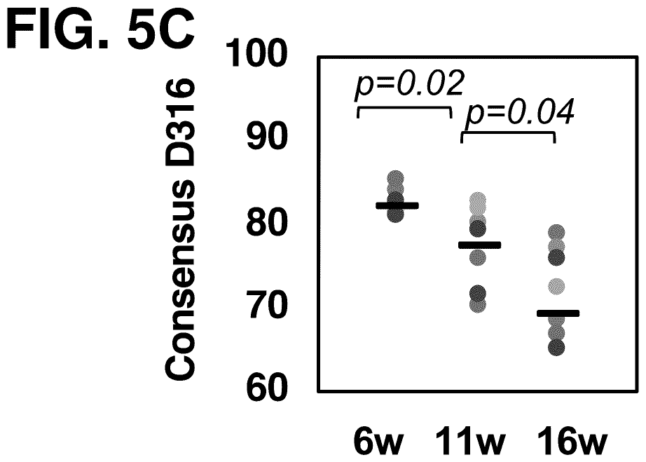

The number of different sequences, or variants, can be calculated as a fractional amount. See FIGS. 4B and 5A. A number of different sequences is needed because, in addition to the approximately 100 major HERV-K Gag sequences, there are other genome sequences and post-transcriptionally mutated sequences that resemble the Gag genes to varying degrees. Sequencing according to the present invention does not exclude these sequences from the major Gag genes. Therefore, a higher complexity of sequence variants indicates increased ERV activation and mutation, which can be calculated as:

Percentage of variants=100×Number of distinct variants/Number of total base pairs in the total gene sequence read.

A higher percentage indicates increased ERV activation and mutation.

Sequences that encode an ORF are needed because after the gene is transcribed into RNA, further mutations can occur and be detected during deep sequencing. See FIGS. 4C, 5B, 6A, 7A, 7B, 7C. The sequences that encode an ORF are extracted and then analyzed for mutations. In embodiments, nucleotide sequences are translated into amino acid sequences to identify sequences that code for an ORF of Gag, ensuring there are no stop codons within the sequences. A percentage of ORFs can be calculated as follows:

Percentage of ORFs=100×Number of sequences coding for an ORF/Number of total sequences.

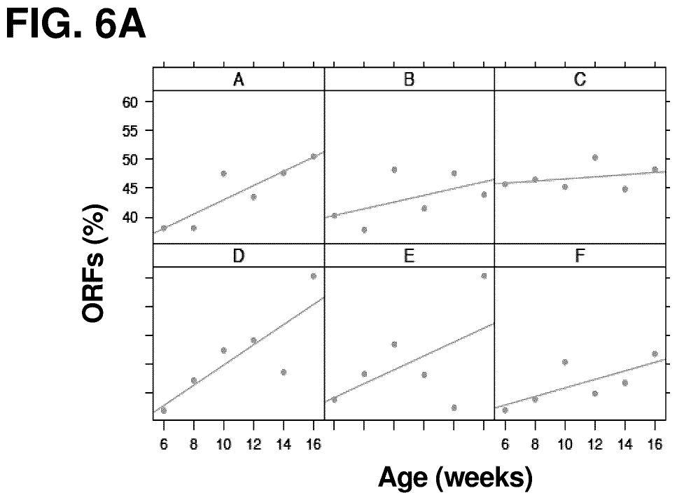

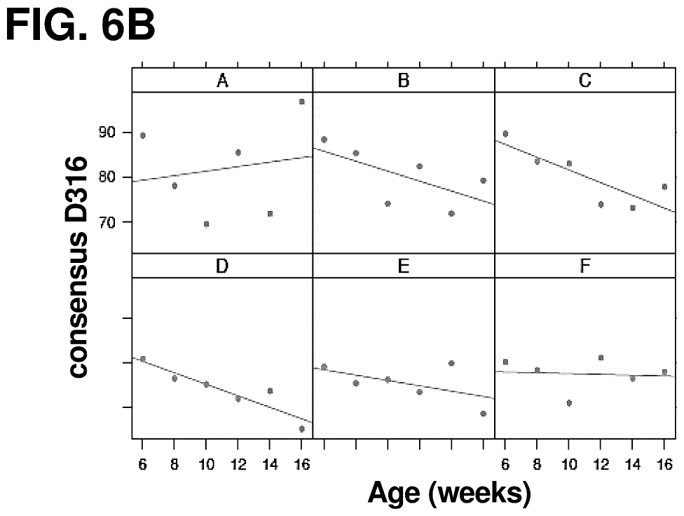

Finally, a consensus score for each nucleotide, or amino acid, of the ORFs can be calculated. See FIGS. 5C and 6B. Individual nucleotides or amino acid residues are mutated unequally, which can be determined by aligning the ORFs and calculating consensus scores. These scores can then be used to predict disease risk and progression. To exclude nucleotide mutations that do not alter amino acid sequences (referred to as anonymous mutations), consensus scores are calculated using amino acid sequences as disease biomarkers. Consensus scores are determined using a scoring matrix that considers the weight of each amino acid residue at its corresponding position in the sequence. Several software and programs are available for sequence alignment to perform this calculation. Advantageously, consensus scores can be quantified as a biomarker of disease progression.

### EXAMPLES

The following non-limiting examples illustrate aspects of the present invention.

### Example 1. Comparing NOD and T1D-Resistant Mice for the Expression of Gag Variants in Islets

To identify short amplicons within the Gag gene, 26 Gag clones derived from islets and 46 genomic Gag sequences were aligned to identify a conserved region of Gag. Primers were designed to produce a PCR amplicon, nt838-1197 (360 bp). Using regular Sanger sequencing, variants on the chromatograms were observed for NOD but not for T1D-resistant B6 islet samples. FIG. 4A illustrates a chromatogram of the nt922-978 region, with double peaks containing sequence variants indicated by arrows. (B/c: Balb/c). Deep sequencing was performed to quantify individual sequence variants within the nt838-1197 amplicon. For each sample, we obtained at least 200,000 sequences and analyzed a minimum of 50,000 matched sequences. By comparing islet samples from NOD and T1D-resistant mouse strains, we observed a significant increase in the percentages of distinct variants in NOD mice, from 11.36% and 11.93% in B6 and Balb/c mice, respectively, to an average of 15.36% in NOD mice (FIG. 4B). Meanwhile, the percentage of sequences encoding ORFs is significantly lower in NOD mice, with an average of 44.91%, compared to 72.21%, 65.81%, and 55.57% in B6, Balb/c, and NCG mice, respectively (FIG. 4C). This suggests that the expression of non-coding “Gag-like” sequences in the islets may be causing the decrease in the percentage of ORFs in NOD mice. Each dot represents one mouse; bars indicate averages for each group; ** indicates p values<0.01. (p value is the probability under the assumption of no effect or no difference (null hypothesis), of obtaining a result equal to or more extreme than what was actually observed.)

### Example 2. Prediction of Prediabetic Stages in NOD Mice

To determine changes in the numbers of Gag variants and ORFs during the prediabetic stages, we compared serum samples from three age groups of NOD mice: 6, 11, and 16 weeks old (8 mice per group). We used deep sequencing analysis of the nt838-1197 amplicon produced in Example 1 to monitor the changes. Percentage of distinct variants (FIG. 5A), percentage of ORFs (FIG. 5B), and consensus score for D316 (FIG. 5C) were calculated and compared among the 3 age groups. (D316 refers to a position in the mouse Gag gene, which is not present in the human HERV-K Gag gene.) The percentages of different variants (FIG. 5A) and ORFs (FIG. 5B) increased steadily from 6 to 11 and then 16 weeks old, whereas the consensus score for one amino acid residue, D316, decreased (FIG. 5C). In other words, there was more variation of the amino acid at position D316 across samples. This suggests that the three scores—the percentage of distinct variants, the percentage of ORFs, and the consensus score for D316—can predict islet destruction and disease progression. The data also indicate that abnormal expression of Gag initially occurs in the islets at a young age. As the disease progresses, Gag sequence variants with ORFs and specific mutations become detectable in the sera, making it a useful biomarker for monitoring islet destruction. ** indicates p values<0.01. Other comparisons (p>0.05) are not shown.

### Example 3. A Longitudinal Analysis of Prediabetic NOD Mice

Serum samples were collected from a group of female NOD mice aged 6 to 20 weeks. Only mice that developed diabetes between 16 and 17 weeks were included in this study. A total of 6 mice (labeled A, B, C, D, E, and F) were used, and serum samples were taken at 6, 8, 10, 12, 14, and 16 weeks. These samples were then analyzed by targeting the nt838-1197 region for deep sequencing. The percentage of ORFs (FIG. 6A) and consensus scores at position D316 (FIG. 6B) were calculated. Linear mixed effects model was used to examine the change of the four markers over time: A: 0.949 (0.227), p<0.001; B: 0.667 (0.314), p=0.042. A significant increase in ORFs over time was observed (p<0.001) (see FIG. 6A). At 10 weeks, a significant increase in ORFs was detected, with an average of 39.51% at 6 weeks increasing to 47.05% at 10 weeks (p=0.006). The peak was reached at 51.68% just before disease onset at 16 weeks. Furthermore, the consensus score of D316 residue was also found to be associated with disease progression (p=0.042) (see FIG. 6B), showing a significant decrease as early as 8 weeks of age (p=0.014 when comparing week 6 vs. week 8). This indicates that both the ORFs and the D316 consensus score are sensitive biomarkers predicting disease progression at the early prediabetic stage.

### Example 4. Amplicon Deep Sequencing of HERV-K Gag Gene and Its Variants

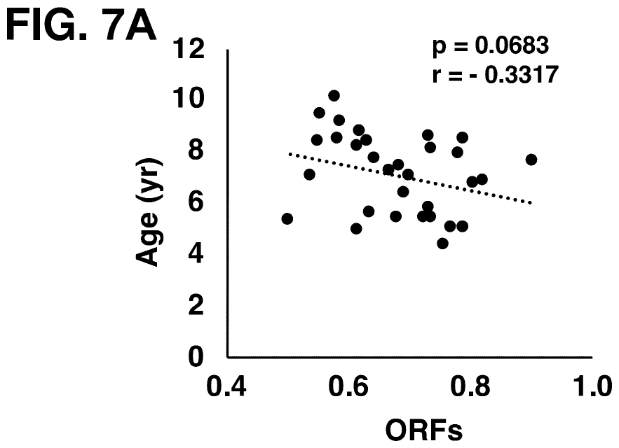

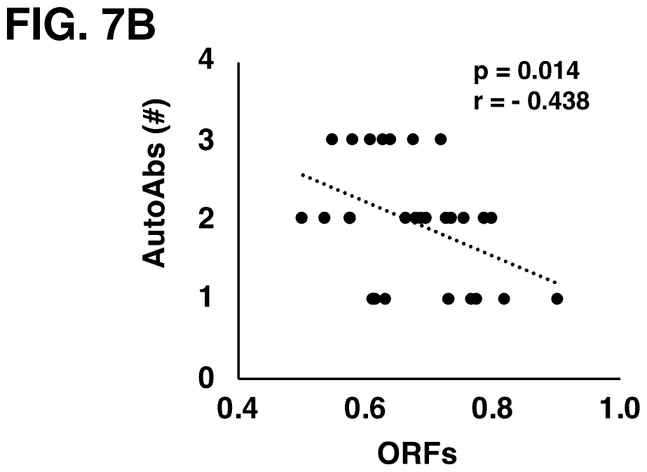

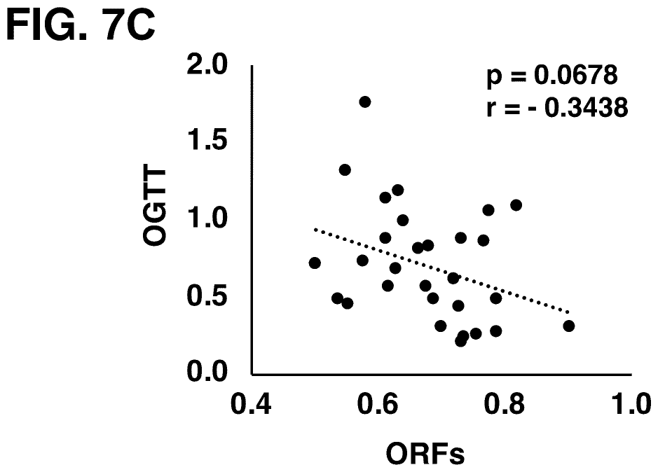

To study human T1D, we analyzed frozen sera obtained from the National Institute of Diabetes and Digestive Kidney Diseases Central Repository (NIDDK-CR). NIDDK-CR provided patients' clinical data, including age, autoantibody, C-peptide, and oral glucose tolerance test (OGTT), both at the visit and during the follow-up periods (every 6 months for up to 5 years). We tested sera (n=31, 100 μl each) collected at the 3rd month after the initial T1D diagnosis, preparing the samples using RNA purification, followed by DNase treatment and cDNA synthesis. To overcome the issue of the low copy number of HERVs, we used a nested PCR protocol. We used one set of primers capable of amplifying all patients' samples to generate an amplicon, nt652-1065 (414 bp), for deep sequencing. We analyzed the follow-up visit data to determine whether the ORFs at the 3rd month of onset could predict disease progression in subsequent visits. The relationship of the % of ORFs with age of disease onset (FIG. 7A), the number of autoantibodies (AutoAbs; see FIG. 7B), and the OGTT results (FIG. 7C) at the 3-month visit were analyzed using a non-parametric Spearman's regression test. We observed a correlation between the ORFs and both the age (see FIG. 7A) and autoantibodies (see FIG. 7B). The percentage of ORFs showed a moderate inverse correlation with the age of T1D onset, as seen in FIG. 7A, whereas single autoantibodies strongly correlated with the increase in ORFs, as shown in FIG. 7B. The percentage of ORFs was significantly higher (p=0.035) in the single autoantibody patients. Additionally, a significant correlation was found between the ORFs and the OGTT30-0′ results (C-peptide changes from 0 to 30 minutes) at the 3-month visit (FIG. 7C). We observed a significant decrease in C-peptide in the ORF-high group at both the 3- and 6- month visits. This suggests that an increase in ORFs is associated with a decrease in islet function.

It should be understood, of course, that the foregoing relates to exemplary embodiments of the invention and that modifications may be made without departing from the spirit and scope of the invention as set forth in the following claims.

What is claimed is:

1. A method of diagnosis of an autoimmune disease using a biomarker, comprising:
collecting a serum sample of a subject;
isolating at least one ribonucleic acid (RNA) from the serum sample;
generating at least one complementary deoxyribonucleic acid (cDNA) from the at least one RNA;
generating at least one polymerase chain reaction (PCR) amplicon from the at least one cDNA using a primer specific for an endogenous retrovirus gene fragment;
sequencing the at least one amplicon utilizing deep sequencing methods to generate at least one nucleotide sequence; and
analyzing the at least one nucleotide sequence, wherein the analyzing includes quantifying at least one score indicative of the autoimmune disease.

2. The method of claim 1, wherein the autoimmune disease is Type 1 Diabetes (T1D).

3. The method of claim 1, wherein the step of generating the at least one cDNA further comprises degrading the at least one RNA to produce fragments of endogenous retroviruses.

4. The method of claim 1, wherein, in the step of generating the at least one PCR amplicon, the endogenous retrovirus gene fragment includes gene fragments having greater than 80% of nucleotides present in SEQ ID NO: 1 in an order identical to that of SEQ ID NO: 1.

5. The method of claim 1, wherein the step of generating the at least one PCR amplicon further comprises:
generating the primer, wherein the primer is configured to bind to a selected region of SEQ ID NO: 1, and wherein the primer is selected from the group consisting of: SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and any combination thereof; and any primer having greater than 80% of nucleotides present in any of SEQ ID NOS: 2, 3, 4, 5, 6, 7, 8, 9, 10, 12, 13, 14, 15, 16, 17, 18, 19, 20, 21, 22, 23, 24, and 25, in an order identical thereto; and
binding the cDNA and the primer.

6. The method of claim 5, wherein the selected region is nt652-1065.

7. The method of claim 5, wherein the selected region is nt308-669.

8. The method of claim 5, wherein the selected region is nt652-1040.

9. The method of claim 1, wherein the at least two sequencing methods include Sanger Sequencing and Amplicon Deep Sequencing.

10. The method of claim 1, wherein the step of analyzing includes aligning nucleotide and protein sequences and wherein the at least one score includes a consensus score of the at least one nucleotide sequence and/or at least one amino acid residue corresponding to the at least one nucleotide sequence.

11. The method of claim 1, wherein the at least one score includes a percentage of sequence variants.

12. The method of claim 1, wherein the at least one score includes a percentage of open reading frames (ORFs).

13. The method of claim 1, further comprising:
providing a digital PCR kit prior to generating the at least one PCR amplicon, wherein the at least one PCR amplicon is operative to amplify the endogenous retrovirus gene fragment;
partitioning the at least one cDNA into a plurality of samples prior to generating the at least one PCR amplicon;
simultaneously amplifying the endogenous retrovirus gene fragment from each of the plurality of samples; and
monitoring and measuring the amplification of the retrovirus gene fragment in real-time.

14. The method of claim 1, wherein the autoimmune disease is selected from the group consisting of Acquired hemophilia, Acromegaly, Agammaglobulinemia, Alopecia Areata, Ankylosing Spondylitis, Anti-NMDA receptor encephalitis, Antiphospholipid Syndrome, Aplastic Anemia, Arteriosclerosis, Autoimmune Addison's disease, Autoimmune Autonomic Ganglionopathy, Autoimmune Encephalitis, Autoimmune encephalitis/Acute disseminated encephalomyelitis (ADEM), Autoimmune gastritis, Autoimmune hemolytic anemia, Autoimmune Hepatitis, Autoimmune hyperlipidemia, Autoimmune Hypophysitis/Lymphocytic hypophysitis, Autoimmune inner ear disease, Autoimmune lymphoproliferative syndrome, Autoimmune myelofibrosis, Autoimmune myocarditis, Autoimmune oophoritis, Autoimmune pancreatitis, Autoimmune polyglandular syndromes, Autoimmune progesterone dermatitis, Autoimmune Retinopathy, Autoimmune sudden sensorineural hearing loss, Balo Disease, Behçet's disease, Birdshot chorioretinopathy, Bullous pemphigoid, Castleman disease, Celiac disease, Chagas disease, Chronic autoimmune urticaria, Chronic inflammatory demyelinating polyneuropathy (CIDP), Churg-Strauss syndrome, Cogan syndrome, Cold agglutinin disease, CREST Syndrome, Crohn's disease, Cronkhite-Canada Syndrome, Cryptogenic organizing pneumonia (COP), Dermatitis herpetiformis, Dermatomyositis, Discoid lupus, Dressler's syndrome, Eczema/Atopic Dermatitis, Endometriosis, Eosinophilic esophagitis/eosinophilic gastroenteritis, Eosinophilic fasciitis, Erythema Nodosum, Essential mixed cryoglobulinemia, Evans syndrome, Fibrosing alveolitis/Idiopathic pulmonary fibrosis (IPF), Giant cell arteritis/temporal arteritis/Horton's disease, Glomerulonephritis, Goodpasture's syndrome/anti-GBM/anti-TBM disease, Granulomatosis with polyangiitis (GPA)/Wegener's granulomatosis, Graves' disease, Guillain-Barrè Syndrome, Hashimoto's Thyroiditis/autoimmune thyroiditis, Henoch-Schonlein purpura, Hidradenitis suppurativa, Hurst's disease/Acute hemorrhagic leukoencephalitis (AHLE), Hypogammaglobulinemia, IgA nephropathy, IgG4-related sclerosing disease (ISD), Immune thrombocytopenia (ITP)/autoimmune thrombocytopenia purpura, Immune-mediated necrotizing myopathy, Inclusion Body Myositis (IBM), Interstitial cystitis, Juvenile Idiopathic Arthritis/Adult-onset Still's Disease, Lambert-Eaton myasthenic syndrome, Lichen Planus, Lichen sclerosus, Linear IgA disease, Lupus Nephritis, Lyme Disease, Ménière's disease, Microscopic polyangiitis (MPA)/ANCA-associated vasculitis, Mixed Connective Tissue Disease, Multiple Sclerosis, Myalgic encephalomyelitis/Chronic fatigue syndrome, Myasthenia Gravis, Neuromyelitis Optica/Devic's disease, Ocular Cicatricial pemphigoid, Palindromic rheumatism, Palmoplantar pustulosis, Paraneoplastic cerebellar degeneration, Paraneoplastic pemphigus, Paroxysmal nocturnal hemoglobinuria, Parry-Romberg syndrome (PRS)/Hemifacial atrophy (HFA)/Progressive facial hemiatrophy, Parsonage-Turner syndrome, Pemphigoid gestationis, Pemphigus foliaceus, Pemphigus Vulgaris, POEMS syndrome, Polyarteritis nodosa, Polymyalgia rheumatica, Polymyositis, Postural orthostatic tachycardia syndrome (POTS), Primary biliary cirrhosis, Primary sclerosing cholangitis, Psoriasis, Psoriatic Arthritis, Pure Red Cell Aplasia, Raynaud's Syndrome, Reactive arthritis, Relapsing polychondritis, Rheumatic Fever, Rheumatoid Arthritis, Sarcoidosis, Schmidt syndrome, Scleritis, Scleroderma, Sjögren Syndrome, Small fiber sensory neuropathy, Sydenham's chorea, Systemic Lupus Erythematosus, Testicular Autoimmunity, Ulcerative Colitis, Undifferentiated Connective Tissue Disease, and Vitiligo.
